(19) United States
(12) Patent Application Publication
(10) Pub. No.: US 2026/0104403 A1
(43) Pub. Date: Apr. 16, 2026

(54) LOAD ESTIMATION METHOD

(71) Applicant: TOYOTA JIDOSHA KABUSHIKI KAISHA, Toyota-shi (JP)

(72) Inventors: Katsuya SASAKI, Nagoya-shi (JP); Ryohei HIROTA, Toyota-shi (JP); Kimihiro NARUSE, Kariya-shi (JP)

(73) Assignee: TOYOTA JIDOSHA KABUSHIKI KAISHA, Toyota-shi (JP)

(21) Appl. No.: 19/096,896

(22) Filed: Apr. 1, 2025

(30) Foreign Application Priority Data
Aug. 15, 2024 (JP) 2024-135532

(51) Int. Cl. G01N 33/28; G07C 5/08

(52) U.S. Cl. CPC G01N 33/28; G07C 5/0808

(57) ABSTRACT

The load estimation method is a method of counting the number of times of activation of the oil pump when the viscosity of the oil discharged from the oil pump is equal to or higher than a threshold value or when the temperature of the oil is equal to or lower than a threshold value, and estimating the thermal load of the oil pump based on the counted number of times of activation.

### CROSS-REFERENCE TO RELATED APPLICATION

This application claims priority to Japanese Patent Application No. 2024-135532 filed on Aug. 15, 2024. The disclosure of the above-identified application, including the specification, drawings, and claims, is incorporated by reference herein in its entirety.

### BACKGROUND

### 1. Technical Field

The present disclosure relates to a load estimation method.

### 2. Description of Related Art

Japanese Unexamined Patent Application Publication No. 6-288215 (JP 6-288215 A), for example, describes a point of correction of operation time of an oil pump in accordance with viscosity of oil, such that a flow rate of the oil on a circulation channel through an oil tank, an oil pan, and an engine, is maintained constant.

### SUMMARY

Although heat is generated by applying electricity to a motor during operation of the oil pump, appropriately comprehending the state of the oil pump is difficult, because there is no method for easily estimating thermal load.

Accordingly, the present disclosure has been made in view of the above problems, and an object thereof is to provide a load estimation method that enables easily estimating a thermal load of an oil pump.

The load estimation method according to the present disclosure is a method that includes counting the number of times of activation of an oil pump, when viscosity of oil that is discharged by the oil pump is no smaller than a threshold value, or when temperature of the oil is no higher than a threshold value, and estimating a thermal load of the oil pump, based on the number of times of activation that is counted.

In the above load estimation method, the viscosity or the temperature may be measured prior to activation of the oil pump.

In the above load estimation method, the viscosity or the temperature may be measured at a position that is downstream of the oil pump in a channel for the oil.

In the above load estimation method, the number of times of activation may be counted for each period from when an ignition switch of a vehicle that is equipped with the oil pump is turned on, until when the ignition switch is turned off.

In the above load estimation method, the thermal load may be estimated based on a frequency distribution of the number of times of activation for each of the periods.

According to the present disclosure, the thermal load of the oil pump can be easily estimated.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a configuration diagram illustrating an example of a vehicle system;

FIG. 2 is a diagram illustrating an example of an activation number of times table;

FIG. 3 is a flow chart illustrating an exemplary operation of a pump ECU (Electronic Control Unit);

FIG. 4 is a block diagram illustrating another example of the vehicle-system and.

FIG. 5 is a flow chart illustrating another exemplary operation of the pump ECU.

### DETAILED DESCRIPTION OF EMBODIMENTS

### Configuration of Vehicle System

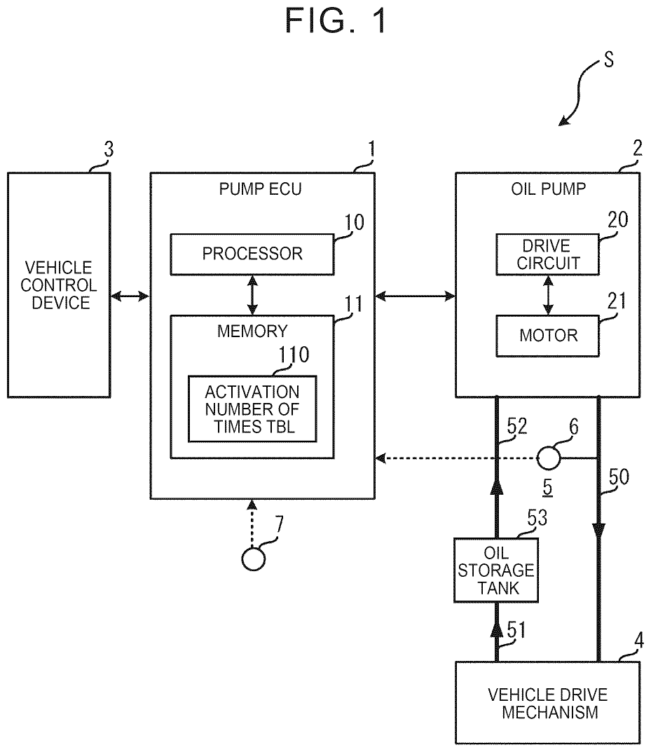

FIG. 1 is a configuration diagram illustrating an example of a vehicle system S. The vehicle system S is mounted on a vehicle such as a hybrid electric vehicle or a battery electric vehicle, and includes a pump ECU 1, an electric oil pump 2, a vehicle control device 3, a vehicle drive mechanism 4, an oil circulation channel 5, a viscosity sensor 6, and an ignition switch 7.

The vehicle drive mechanism 4 includes various devices and components (not shown) such as an engine, a motor, a transmission, and a gear for driving the vehicle. At least a part of the vehicle drive mechanism 4 is supplied with oil from the oil pump 2 via the circulation channel 5. Thus, at least a part of the vehicle drive mechanism 4 is lubricated or cooled.

The circulation channel 5 is an example of an oil channel. The circulation channel 5 includes a supply path 50, a discharge path 51, a suction path 52, and an oil storage tank 53. The supply path 50 is disposed on the downstream side of the oil pump 2, and the oil discharged from the oil pump 2 flows through the supply path 50 and is supplied to the vehicle drive mechanism 4.

The discharge path 51 is disposed downstream of the vehicle drive mechanism 4, and oil used for lubrication and cooling of at least a part of the vehicle drive mechanism 4 is discharged from the discharge path 51. The downstream end of the discharge path 51 is connected to the oil storage tank 53. The oil storage tank 53 is, for example, an oil pan, and stores the oil discharged from the discharge path 51.

The suction path 52 is disposed upstream of the oil pump 2, and the oil pump 2 sucks oil through the suction path 52. Thus, the oil circulates between the oil pump 2 and the vehicle drive mechanism 4 via the circulation channel 5.

A viscosity sensor 6 for detecting the viscosity of oil is provided in the suction path 52. The viscosity sensor 6 outputs the detected viscosity of the oil at the position upstream of the oil pump 2 in the circulation channel 5 to the pump ECU 1.

The oil pump 2 includes a drive circuit 20 and a motor 21. The oil pump 2 sucks the oil from the suction path 52 by the rotation of the motor 21 and discharges the oil to the supply path 50. The drive circuit 20 includes an inverter or the like and drives the motor 21. The drive circuit 20 is controlled by a pump ECU 1. The oil pump 2 and the vehicle drive mechanism 4 operate when the ignition switch 7 is on, and stop when the ignition switch 7 is off.

The pump ECU 1 is an exemplary computer and includes a processor 10 such as CPU (Central Processing Unit) and memories 11 such as ROM (Read Only Memory) and RAM (Random Access Memory). The processor 10 executes a series of processes in a predetermined sequence in accordance with a program stored in the memory 11. The pump ECU 1 may be mounted inside the same housing as the vehicle control device 3. In addition, the pump ECU 1 may be used for the process by sharing CPU, ROM and RAM of the vehicle control device 3.

The processor 10 activates and stops the oil pump 2 in accordance with a request from the vehicle control device 3. The vehicle control device 3 calculates a required torque required for the vehicle in accordance with an operation of the driver, and controls an output torque of the engine based on the required torque.

The processor 10 outputs an activation signal to the drive circuit 20 of the oil pump 2. Further, the processor 10 outputs the target rotational speed of the motor 21 of the oil pump 2 to the drive circuit 20 together with the activation signal. The drive circuit 20 drives the motor 21 so that the rotational speed of the motor 21 becomes the target rotational speed in accordance with the activation signal. As a result, the motor 21 is operated, and the oil is discharged from the oil pump 2.

Each time the oil pump 2 is activated, the drive circuit 20 and the motor 21 generate heat by energization. At this time, the higher the viscosity of the oil in the circulation channel 5, the larger the output torque of the motor 21, and thus the larger the calorific value. As a result, a thermal load of the oil pump 2 is generated. When the activation of the oil pump 2 is continuously performed within a short period of time, heat is generated again before the drive circuit 20 and the motor 21 are sufficiently cooled, so that a thermal load is accumulated.

For example, in the oil pump 2, when the oil has a high viscosity, the rotational speed of the motor 21 may not reach the target rotational speed. The pump ECU 1 instructs the oil pump 2 to retry the activation process. At this time, the pump ECU 1 repeats the activation process many times in order to re-output the activation signal to the oil pump 2. As a result, the oil pump 2 is continuously activated, and the thermal load is significantly increased.

Therefore, when the viscosity of the oil discharged from the oil pump 2 is equal to or greater than the threshold value, the processor 10 counts the number of times of activation of the oil pump 2, and estimates the thermal load of the oil pump 2 based on the counted number of times of activation. For example, the processor 10 records the number of times of activation of the oil pump 2 in the number-of-times table (TBL) 110 held in the memory 11, and estimates the thermal loads from the number-of-times TBL110 of activation. According to the load estimation method, the thermal load can be easily estimated as compared with, for example, a case where a plurality of sensors for detecting the amount of heat are provided in the oil pump 2 to calculate the thermal load.

The processor 10 measures the viscosity before the activation of the oil pump 2. Therefore, the processor 10 can more reliably determine that the oil pump 2 is activated when the oil has a high viscosity, as compared with, for example, a case where the viscosity of the oil stirred by the activation of the oil pump 2 is measured.

The processor 10 measures the viscosity at a position downstream of the oil pump 2 in the oil circulation channel 5 by the viscosity sensor 6. Therefore, the processor 10 can more reliably determine that the oil pump 2 is activated when the oil has a high viscosity by using the viscosity at the point where the oil pump 2 is discharged and pushed in, as compared with, for example, a case where the viscosity at the position upstream of the oil pump 2 is measured. The viscosity sensor 6 is not limited to the position on the downstream side, and may be provided, for example, in a position on the upstream side of the oil pump 2, or in a position on the middle-stream supply path 50 or in the oil storage tank 53.

The processor 10 counts the number of times the ignition switch 7 is turned on and off, that is, counts the number of times of activation. Therefore, the processor 10 can count the thermal load of the oil pump 2 when the ignition switch 7 is continuously operated without being turned off as the number of times of activation. Therefore, it is possible to estimate the thermal load with high accuracy as compared with, for example, a case where the number of times of activation at predetermined time intervals is counted. The processor 10 is not limited to counting for each trip, and may count the number of times of activation at predetermined time intervals.

### Example of Thermal Load Estimation

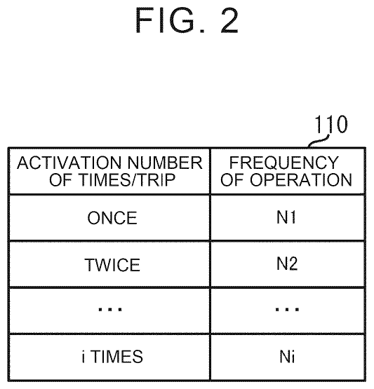

FIG. 2 is a diagram illustrating an example of the activation number of times table 110. In the activation number of times TBL110, the activation number of times for each trip and the operation frequency are registered. For example, the operation frequency where the number of times of activation is 1 is N1, the operation frequency where the number of times of activation is 2 is N2, and the operation frequency where the number of times of activation is i is Ni (N1,N2,Ni,i: positive integer).

$$Lt = K 1 \times W 1 \times N 1 + K 2 \times W 2 \times N 2 + … + K i \times W i \times N i \qquad ( 1 )$$

The processor 10 calculates the thermal loading Lt of the oil pump 2, for example, from the above equation (1) based on the activation number of times TBL110. In Expression (1), K1, K2, . . . , Ki is a temperature coefficient of oil when the number of times of activation is 1, 2, . . . , and i, respectively. In Expression (1), W1, W2, . . . , Wi is a weighting factor when the number of times of activation is 1, 2, . . . , and i, respectively. The temperature coefficient K1, K2, . . . , Ki is an increase amount of the heat amount according to the number of times of activation, and the weight coefficient is a coefficient related to the heat increase according to the structural design of the oil pump 2.

In this way, the processor 10 estimates the thermal load based on the frequency distribution of the number of times of activation per trip. Therefore, according to the above-described load estimation methods of the pump ECU 1, it is possible to estimate the thermal load with high accuracy in accordance with the amount of heat generated by continuously activating the oil pump 2 in trip units. Note that the thermal load estimated in this way can be used for notification of the replacement time and the life of the oil pump 2, drive control of the oil pump 2, and the like.

### Operation of the Pump ECU

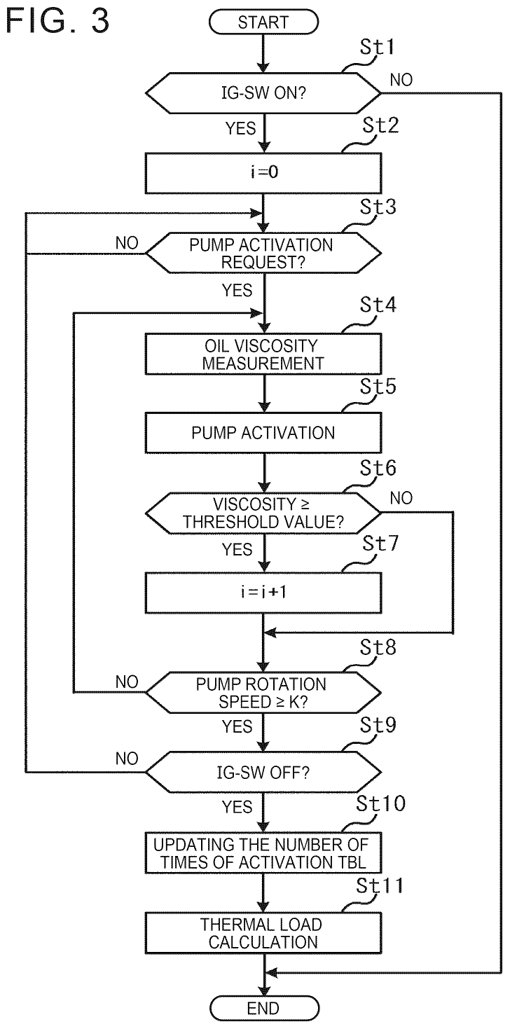

FIG. 3 is a flow chart illustrating an exemplary operation of the pump ECU 1. This operation is repeatedly performed, for example, when the processor 10 executes a program in the memory 11. This operation is an example of a load estimation method.

First, the pump ECU 1 determines whether the ignition switch (IG-SW) 7 is on (St1). When the ignition switch 7 is off (No of St1), this operation ends.

When the ignition switch 7 is on (Yes of St1), the number of times i of activation in the new trip is set to 0 (St2). Next, the pump ECU 1 determines whether or not there is a demand for activation of the oil pump 2 from the vehicle control device 3 (St3). If there is no activation request (No of St3), St3 operation is re-executed.

If there is an activation request (Yes of St3), the pump ECU 1 measures the viscosity of the oil by means of the viscosity sensor 6 (St4). Next, the pump ECU 1 activates the oil pump 2 by outputting an activation instruction to the drive circuit 20 (St5). In this way, the viscosity measurement is performed before the activation of the oil pump 2.

The pump ECU 1 then compares the viscosity as measured by the viscosity sensor 6 with the threshold value (St6). When the viscosity≥threshold value is satisfied (Yes of St6), the pump ECU 1 adds 1 to the number of times of activation i (St7). When the viscosity<threshold value is satisfied (No of St6), the operation of St7 is not executed. In this way, the pump ECU 1 counts the number of times of activation i of the oil pump 2 when the oil is highly viscous. The number of times of activation i is counted up when the start instruction is output. The viscosity threshold value is determined according to the design, experiment, simulation result, or the like of the oil pump 2.

Next, the pump ECU 1 compares the rotational speed of the motor 21 notified from the drive circuit 20 with a predetermined value K (St8). When the rotational speed<K is satisfied (No of St8), the operations after St4 are executed again.

When the rotational speed≥K is satisfied (Yes of St8), the pump ECU 1 determines whether the ignition switch 7 is off (St9). When the ignition switch 7 is on (No of St9), the operations after St3 are executed again.

When the ignition switch 7 is off (Yes of St9), the pump ECU 1 updates the activation number of times TBL110 (St10) and calculates the thermal loading (St11). In this way, the pump ECU 1 operates.

### Another Embodiment

In the above embodiment, the viscosity of the oil is measured by the viscosity sensor 6 in the pump ECU 1, but is not limited thereto. Since the viscosity of the oil varies depending on the temperature, the pump ECU 1 may use a temperature sensor instead of the viscosity sensor 6.

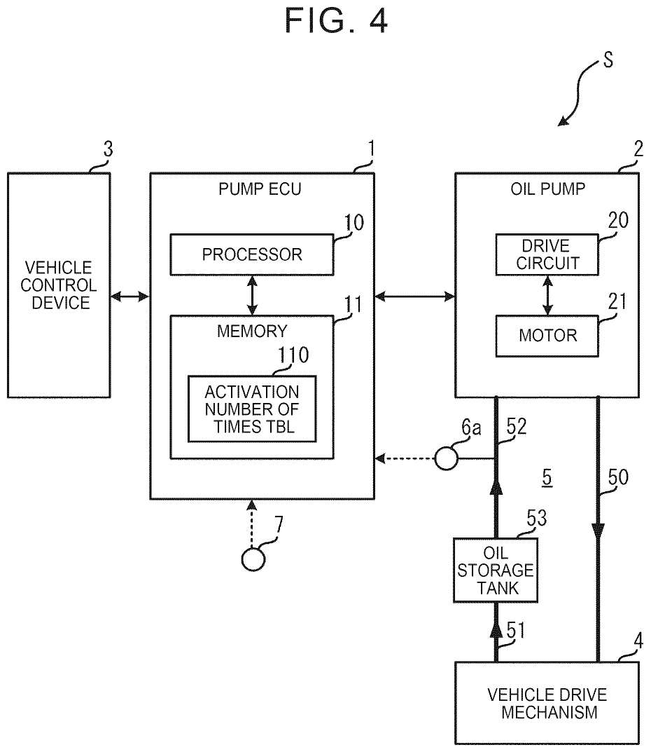

FIG. 4 is a configuration diagram illustrating another example of the vehicle system S. In FIG. 4, the same components as those in FIG. 1 are denoted by the same reference numerals, and the description thereof will be omitted. In the present embodiment, a temperature sensor 6a is provided in the suction path 52 instead of the viscosity sensor 6. The temperature sensor 6a detects the temperature of the oil flowing through the suction path 52. The position of the temperature sensor 6a is preferably provided at a position downstream of the oil pump 2 in the same manner as the viscosity sensor 6. However, the present disclosure is not limited thereto, and may be provided, for example, at a position on the upstream side of the oil pump 2, or at a position on the medium-flow supply path 50 or in the oil storage tank 53. The temperature-sensor 6a outputs the detected value to the pump ECU 1.

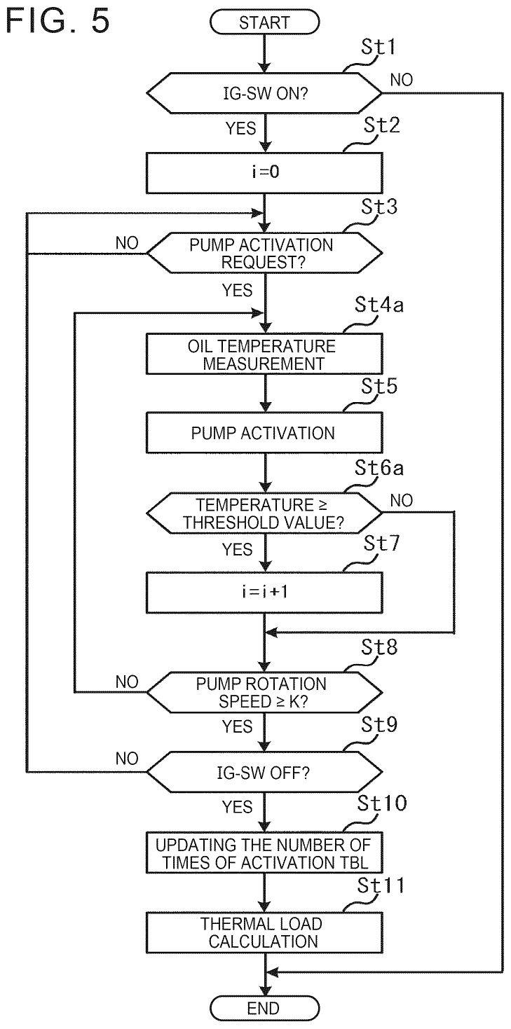

FIG. 5 is a flow chart illustrating another exemplary operation of the pump ECU 1. In FIG. 4, operations common to those in FIG. 3 are denoted by the same reference numerals, and description thereof will be omitted.

If there is an activation requirement (Yes of St3), the pump ECU 1 measures the temperature of the oil by means of a temperature sensor 6a (St4a). After activation (St5) of the oil pump 2, the pump ECU 1 compares the temperature measured by the temperature sensor 6a with the threshold values (St6a). If the temperature≤threshold value is satisfied (Yes of St6a), the pump ECU 1 adds 1 to the number of times of activation i (St7). When the temperature>threshold value is satisfied (No of St6a), St7 operation is not executed.

In this way, the pump ECU 1 counts the activation number of times of the oil pump 2 when the temperature of the oil is equal to or lower than the threshold value. In the properties of oil, the lower the temperature, the higher the viscosity. For this reason, the pump ECU 1 can also determine that the oil is highly viscous by determining the temperature as described above. The threshold value of the temperature is determined according to the design, experiment, simulation result, or the like of the oil pump 2. It should be noted that, although the load estimation method of the present embodiment is executed by a pump ECU 1, the present embodiment may be executed by hardware such as ASIC (Application Specified Integrated Circuit). In addition, although the vehicle system S including the viscosity sensor 6 or the temperature sensor 6a has been exemplified, the vehicle system S may include only one of the viscosity sensor 6 and the temperature sensor 6a because the viscosity of the oil shows a property of uniquely changing depending on the temperature.

The above-described embodiments are preferred embodiments of the present disclosure. However, the present disclosure is not limited thereto, and various modifications can be made without departing from the gist of the present disclosure.

What is claimed is:

1. A load estimation method, comprising:
counting the number of times of activation of an oil pump, when viscosity of oil that is discharged by the oil pump is no smaller than a threshold value, or when temperature of the oil is no higher than a threshold value; and
estimating a thermal load of the oil pump, based on the number of times of activation that is counted.

2. The load estimation method according to claim 1, wherein the viscosity or the temperature is measured prior to activation of the oil pump.

3. The load estimation method according to claim 2, wherein the viscosity or the temperature is measured at a position that is downstream of the oil pump in a channel for the oil.

4. The load estimation method according to claim 1, wherein the number of times of activation is counted for each period from when an ignition switch of a vehicle that is equipped with the oil pump is turned on, until when the ignition switch is turned off.

5. The load estimation method according to claim 4, wherein the thermal load is estimated based on a frequency distribution of the number of times of activation for each of the periods.
